(19) United States
(12) Patent Application Publication
(10) Pub. No.: US 2015/0217075 A1
(43) Pub. Date: Aug. 6, 2015

(54) OXYGEN MASK WITH MEANS TO SAMPLE EXPIRED GASES

(71) Applicant: Anyaneya, LLC, Colleyville, TX (US)

(72) Inventor: Lakshmi Deepak Nair, Colleyville, TX (US)

(73) Assignee: Anyaneya, LLC, Colleyville, TX (US)

(21) Appl. No.: 14/171,996

(22) Filed: Feb. 4, 2014

(51) Int. Cl. A61M 16/08; A61B 5/097; A61M 16/00; A61M 16/10; A61B 1/00; A61B 5/083; A61M 16/06

(52) U.S. Cl. CPC A61M 16/085; A61B 5/0836; A61B 5/097; A61M 16/0616; A61M 16/1005; A61B 1/00154; A61M 16/0003; A61M 2016/103; A61M 2210/0618; A61M 2230/432; A61M 2202/0208; A61M 2210/0625; G01N 33/004

(57) ABSTRACT

An oxygen mask for delivering oxygen to a patient comprises a mask body adapted to substantially cover a patient's nose and mouth and having a peripheral edge adapted to substantially seal against the patient's face and forming a compartment, an oxygen inflow port defined in the mask body, an L-shaped connector coupled to the oxygen inflow port, an oxygen conduit coupled to the L-shaped connector and having access to the compartment in the mask body via the oxygen inflow port, the oxygen conduit adapted to conduct oxygen flow from an oxygen supply to the compartment in the mask body, and an expired gas sampling tube coupled to the mask body and having access to the compartment in the mask body, the expired gas sampling tube having a perforated segment with a plurality of perforations attached to an interior surface of the mask body, the expired gas sampling tube adapted to couple to an instrument operable to monitor the expired gases.

### FIELD

The present disclosure generally relates to medical devices, and is related more specifically to an oxygen mask with means to sample expired gases.

### BACKGROUND

During certain medical procedures, the patient often requires assistance in receiving life-sustaining oxygen. An oxygen mask, typically worn over the patient's nose and mouth, is commonly used to deliver oxygen from a storage tank to the patient. Alternatively, a nasal cannula is often used for oxygen delivery. However, for certain patients the amount of oxygen delivered by a nasal cannula is often inadequate. Therefore, the oxygen mask is the preferred method of providing supplemental oxygen to a patient in many instances.

End-tidal carbon dioxide (ETCO₂) measurement using a capnograph to monitor a patient's respiration is a gold standard of care mandated for procedures where anesthesia is administered. ETCO₂ monitoring has been shown to be more effective than clinical judgment alone in early detection of adverse respiratory events such as hypoventilation, esophageal intubation, and circuit disconnection, thereby assisting the anesthesiologist in taking preventative measures to avoid injury to the patient. For these and other reasons, the American Society of Anesthesiologists' Standards for Basic Anesthetic Monitoring of ETCO₂ became effective Jul. 1, 2011. A continually viewable quantitative end-tidal CO₂ waveform and/or digital readout is currently the de facto standard when general anesthesia is administered or when moderate to deep sedation is induced during certain medical procedures.

### SUMMARY

In an embodiment of the present disclosure, an oxygen mask for delivering oxygen to a patient comprises a mask body adapted to substantially cover a patient's nose and mouth and having a peripheral edge adapted to substantially seal against the patient's face and forming a compartment, an oxygen inflow port defined in the mask body, an L-shaped connector coupled to the oxygen inflow port, an oxygen conduit coupled to the L-shaped connector and having access to the compartment in the mask body via the oxygen inflow port, the oxygen conduit adapted to conduct oxygen flow from an oxygen supply to the compartment in the mask body, and an expired gas sampling tube coupled to the mask body and having access to the compartment in the mask body, the expired gas sampling tube having a perforated segment defining a plurality of perforations attached to an interior surface of the mask body, the expired gas sampling tube adapted to couple to an instrument operable to monitor the expired gases.

In another embodiment of the present disclosure, a mask for delivering oxygen to a patient comprises a mask body adapted to substantially cover a patient's nose and having a peripheral edge adapted to substantially seal against the patient's face and forming a compartment, an oxygen inflow port defined in the mask body, an oxygen conduit coupled to the oxygen inflow port and having access to the compartment in the mask body, the oxygen conduit adapted to conduct oxygen flow from an oxygen supply to the compartment in the mask body, and an expired gas sampling tube coupled to the mask body and having access to the compartment in the mask body, the expired gas sampling tube having a perforated segment defining a plurality of perforations and generally positioned proximate to the patient's nose and mouth, the expired gas sampling tube adapted to couple to an instrument operable to monitor the expired gases.

In another embodiment of the present disclosure, an oxygen mask comprises a mask body adapted to substantially cover a patient's nose and mouth and having a peripheral edge adapted to substantially seal against the patient's face and forming a compartment, an oxygen inflow port defined in the mask body, an oxygen conduit coupled to the mask body via the oxygen inflow port, the oxygen conduit adapted to conduct oxygen flow from an oxygen supply to the compartment in the mask body, at least one vent opening defined laterally in the mask body, and an expired gas sampling tube gaining access to the compartment in the mask body via the at least one lateral vent opening, the expired gas sampling tube having a perforated segment defining a plurality of perforations and attached to an interior surface of the mask body proximate to the patient's nose and mouth, the expired gas sampling tube adapted to conduct expired gases from the patient to an instrument operable to make measurements of the expired gases.

### DESCRIPTION OF THE DRAWINGS

FIG. 1 is a front perspective view of a patient wearing a first exemplary embodiment of an oxygen mask with means to sample expired gases according to the teachings of the present disclosure;

FIG. 2 is a front perspective view of the first exemplary embodiment of an oxygen mask with means to sample expired gases according to the teachings of the present disclosure;

FIG. 3 is a side view of the first exemplary embodiment of an oxygen mask with means to sample expired gases according to the teachings of the present disclosure;

FIG. 4 is a front perspective view of a patient wearing a second exemplary embodiment of an oxygen mask with means to sample expired gases according to the teachings of the present disclosure;

FIG. 5 is a front perspective view of the second exemplary embodiment of an oxygen mask with means to sample expired gases according to the teachings of the present disclosure; and

FIG. 6 is a side view of the second exemplary embodiment of an oxygen mask with means to sample expired gases according to the teachings of the present disclosure;

### DETAILED DESCRIPTION

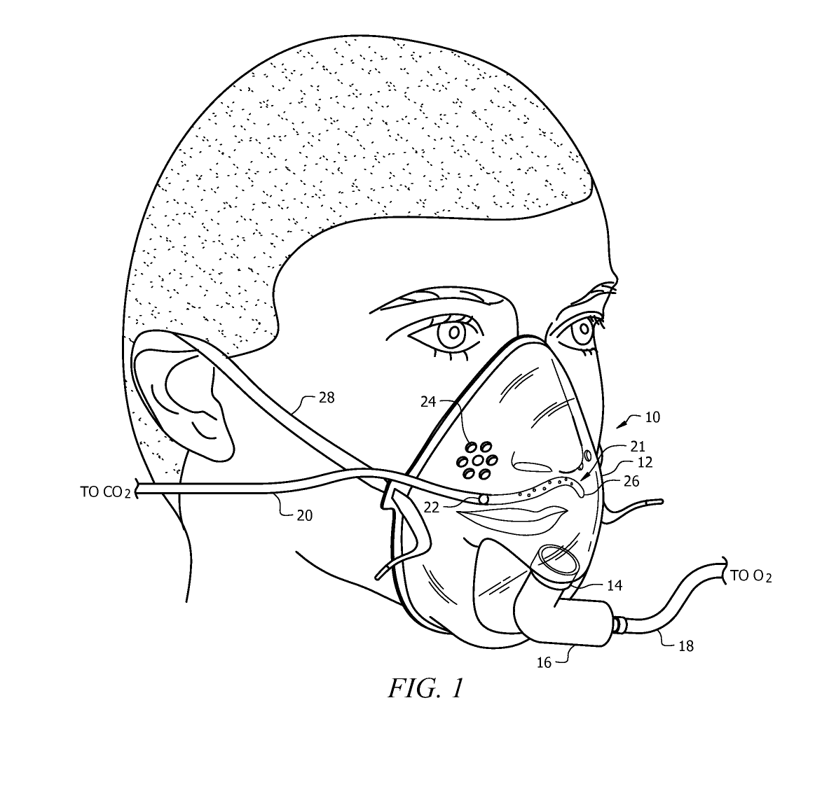

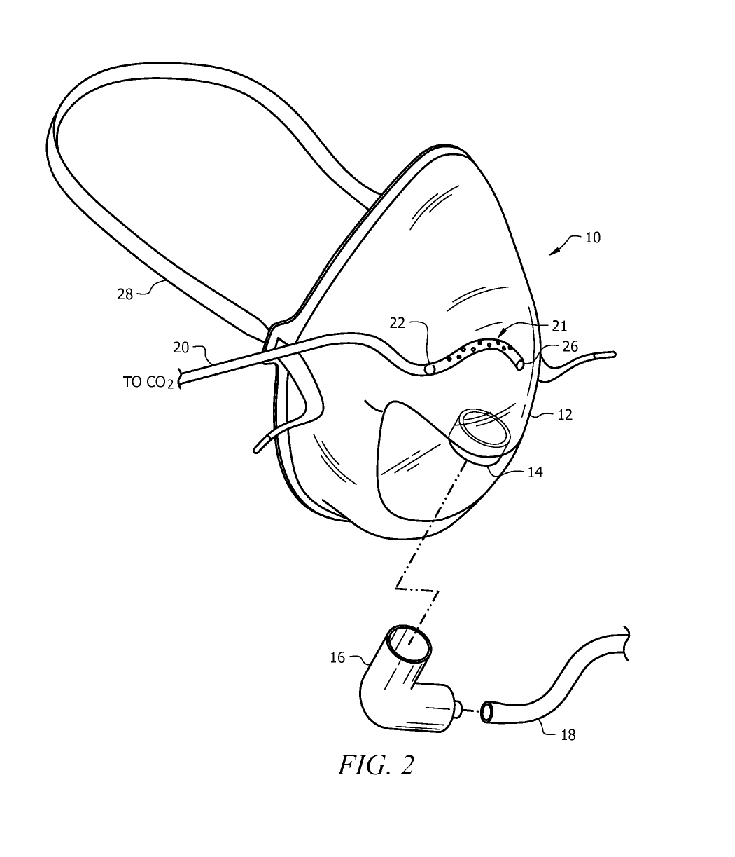

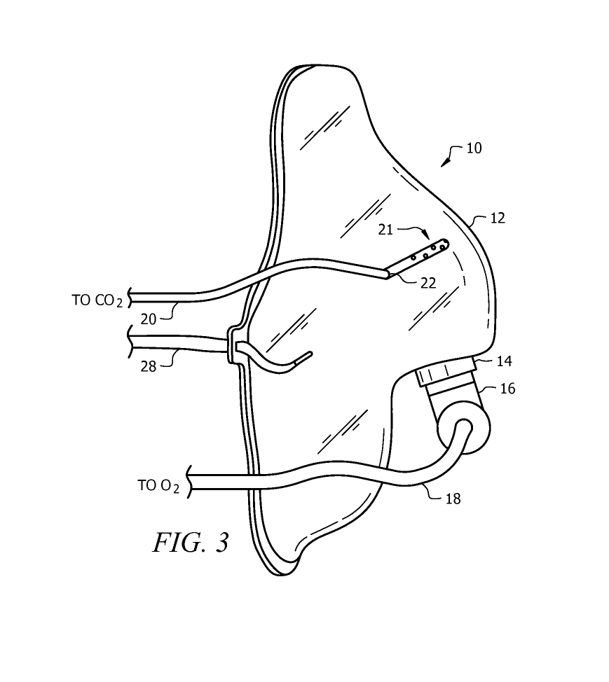

FIGS. 1-3 are illustrative of various views of a first exemplary embodiment of an oxygen mask 10 with means to sample expired gases according to the teachings of the present disclosure. The oxygen mask 10 is generally concave in shape and adapted to cover at least the nose and mouth of the patient and define a compartment therein where oxygen reserves reside. The peripheral edges of the mask 10 are adapted to adhere to the patient's face to form a fairly good seal to keep the oxygen inside the mask 10. The oxygen mask 10 may generally follow the facial profile of the patient such as forming a “nose” portion 12. An oxygen inflow port 14 is defined in the nostril region of the “nose” portion 12 of the mask 10 where an L-shaped or elbow connector 16 is adapted to connect to the oxygen inflow port 14. The L-shaped connector 16 is further connected to an oxygen conduit 18 that leads to an oxygen supply such as an oxygen tank, for example. The L-shaped connector 16 forms an air-tight connection with the oxygen inflow port 14 and the oxygen conduit 18. The location and angle of the oxygen inflow port 14 naturally direct oxygen flowing into the mask toward the patient's nostrils and mouth to further facilitate the patient's breathing. The L-shaped connector 16 also enables the oxygen conduit 18 to connect to the oxygen source typically placed to the side or head of the patient's bed with a decreased risk of forming bends and kinks in the oxygen conduit. The L-shaped connector 16 may be adjusted so that the oxygen conduit 18 may lead from the patient's mask on either side to better adapt to the placement of the oxygen supply relative to the patient's head.

Positioned within the compartment in the nose portion 12 is an expired gas sampling tube 20 adapted to receive carbon dioxide (CO2) exhaled by the patient and conduct it to a capnograph or another form of monitoring device. The capnograph is an electronic device that is capable of measuring the concentration or partial pressure of CO2 in the respiratory gases, and provide output information about the patient's respiratory and circulatory status. Capnography is effective in providing a rapid and reliable method to detect life-threatening conditions in the patient. The expired gas sampling tube 20 is preferably attached at one or more points to an inside surface of the nose portion 12 and is preferably positioned between the patient's nostrils and mouth to sample exhaled gases from both sources.

The expired gas sampling tube 20 is a length of tubing with a perforated segment 21 accommodated inside the mask 10 having a plurality of perforations adapted to intake expired gases from the patient's nose and mouth with increased sensitivity. The expired gas sampling tube 20 may enter one side (e.g., right side) of the mask 10 via a small port 22 or through one or more existing openings such as the vent openings 24 defined on one side of the mask 10. The perforations may be arranged along the segment of the expired gas sampling tube 20 within the nose compartment in a predetermined pattern or they may be randomly distributed. The size or sizes of the perforations and the size of the tubing are preferably optimized for expired gas sampling. One end 26 of the expired gas sampling tube 20 is preferably open, but may be closed off, and the second end of the expired gas sampling tube 20 is directly or indirectly connected to the capnography equipment.

The mask 10 may be secured to a patient's face by a strap or elastic cord 28 attached to the mask 10. The oxygen mask 10 is preferably manufactured of a transparent material to enable a clear and unobstructed view. The mask material is preferably pliable that can easily conform to the patient's facial profile. The oxygen mask may be made of vinyl, plastic, silicone, rubber, or other suitable materials.

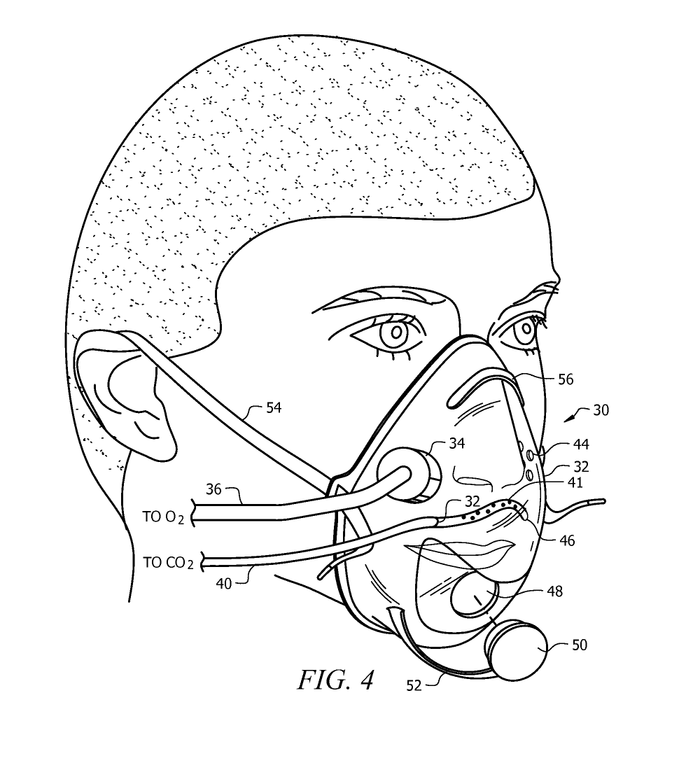

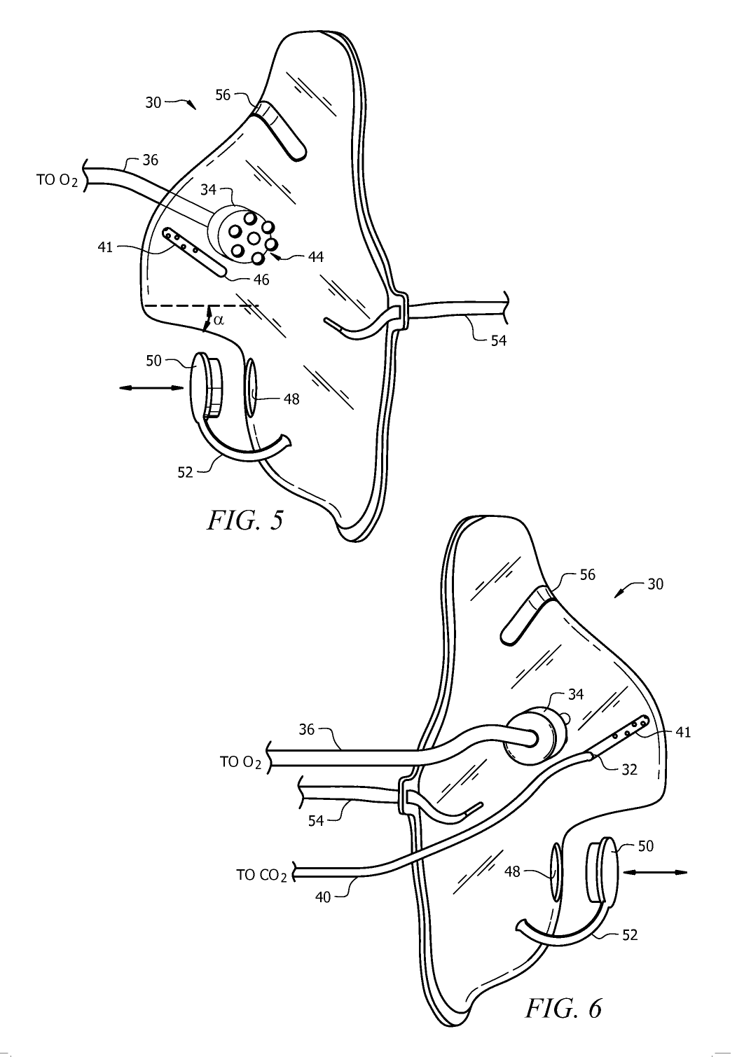

FIGS. 4-6 are illustrative of various views of a second exemplary embodiment of an oxygen mask 30 with means to sample expired gases according to the teachings of the present disclosure. The oxygen mask 30 is adapted to form a compartment that covers the nose and mouth of the patient. The peripheral edges of the mask 30 are adapted to adhere to the patient's face to form a good seal to keep the oxygen inside the mask 30. The oxygen mask 30 may include a “nose” portion 32. An oxygen inflow port 34 is defined on one side (e.g., right side shown) of the mask 30, and an oxygen delivery tube or conduit 36 is adapted to connect to the oxygen inflow port 34. The oxygen conduit 36 is further connected to an oxygen supply such as an oxygen tank, for example. The position of the oxygen inflow port 34 naturally directs oxygen flowing into the mask 30 toward the patient's nostrils and mouth to further facilitate the patient's breathing. The side placement of the inflow port 34 on the mask 30 enables the oxygen conduit 36 to connect to the oxygen source typically placed to the side or head of the patient's bed to have a decreased risk of forming bends and kinks in the conduit. Although not shown explicitly, an L-shaped connector such as the one shown in FIGS. 1-3 may be used to couple the oxygen conduit 36 to the inflow port 34.

Positioned within the compartment in the nose portion 32 is an expired gas sampling tube 40 adapted to receive carbon dioxide (CO2) exhaled by the patient and supply it to a capnograph or another CO2 measurement device. The expired gas sampling tube 40 is preferably attached at one or more points to an inside surface of the mask in the nose portion 32 and is preferably positioned mid-point between the patient's nostrils and mouth. The expired gas sampling tube 40 is a length of tubing with a portion 41 accommodated inside the mask 30 with a plurality of perforations adapted to sample expired gases from the patient's nose and mouth with increased sensitivity. The expired gas sampling tube 40 may enter one side (e.g., right side) of the mask 30 via a small port 32 or through one or more of the existing openings such as the vent openings 44 defined on one side of the mask 30. The perforations may be arranged along the segment of the expired gas sampling tube 40 within the nose compartment in a predetermined pattern or they may be randomly distributed. The size or sizes of the perforations and the tubing are preferably optimized for expired gas sampling. One end 46 of the expired gas sampling tube 40 is preferably open, but may be closed off, and the second end of the expired gas sampling tube 40 is directly or indirectly connected to the capnography equipment.

The oxygen mask 30 further includes a central scope opening or port 48 generally defined below the nose portion 32 and proximate to the general location of the patient's mouth. The central port 48 is sized and adapted to accommodate instruments requiring access to the patient's mouth to obtain views of the patient's internal organs and other structures, such as bronchoscopes, simple endoscopes, endoscopic retrograde cholangiopancreatography (ERCP) endoscopes, and the like. A plug 50 attached to the mask 30 by a short and flexible strap 52 is provided to seal off the central port when such access route is not needed, such as prior to or after the procedure.

The oxygen mask 30 may be secured to a patient's face by a strap or elastic cord 54 attached to the mask 30. The mask 30 is preferably equipped with a nose clip 56 that may be used to better conform and situate the mask 30 to the patient's face. The nose clip 56 is typically made of a pliable metal that can be easily manipulated and adjusted. The mask 30 is also preferably made of a flexible or pliable material that can easily conform to the patient's facial profile. The oxygen mask may be made of vinyl, plastic, silicone, rubber, or other suitable materials.

Although the masks shown herein are of the type that includes a “nose” portion, the teachings of the present disclosure are applicable to masks with a simple domed profile. The expired gas sampling tube is generally attached to the inside surface of the mask at one or more locations so that the perforated segment is positioned proximate to the patient's nose and mouth when the mask is worn by a patient. Preferably the perforated segment is placed at a mid-point between the patient's nose and mouth. The remaining segment of the expired gas sampling tube may be attached to the interior surface of the mask body until the tube exits the mask.

The entry site of the expired gas sampling tube is preferably on the right side of the mask, as it is customary to position the patient on his/her left side (lateral position) for certain medical procedures, including gastrointestinal procedures, such as endoscopy or colonoscopy. However, the teachings of the disclosure herein are similarly applicable to masks with the expired gas sampling tube entering the mask on the left side.

The expired gas sampling tube may gain access to the compartment within the mask body via a dedicated opening, one of the plurality of vent openings, or alternatively via the periphery edge of the mask body. The sampling tube may be attached to the inside surface of the mask body to secure it and ensure proper position of the perforated segment proximate to the patient's nose and mouth.

Although the embodiments of the oxygen mask described herein are of the type that is worn over a patient's nose and mouth, the innovations can also be applied to those masks that are worn over the entire face of the patient. The masks can be sized and proportioned to patients of various sizes, such as adult and children sizes.

The features of the present invention which are believed to be novel are set forth below with particularity in the appended claims. However, modifications, variations, and changes to the exemplary embodiments described above will be apparent to those skilled in the art, and the oxygen mask described herein thus encompasses such modifications, variations, and changes and are not limited to the specific embodiments described herein.

What is claimed is:

1. An oxygen mask for delivering oxygen to a patient, comprising:
a mask body adapted to substantially cover a patient's nose and mouth and having a peripheral edge adapted to substantially seal against the patient's face and forming a compartment;
an oxygen inflow port defined in the mask body;
an L-shaped connector coupled to the oxygen inflow port;
an oxygen conduit coupled to the L-shaped connector and having access to the compartment in the mask body via the oxygen inflow port, the oxygen conduit adapted to conduct oxygen flow from an oxygen supply to the compartment in the mask body; and
an expired gas sampling tube coupled to the mask body and having access to the compartment in the mask body, the expired gas sampling tube having a perforated segment with a plurality of perforations attached to an interior surface of the mask body, the expired gas sampling tube adapted to couple to an instrument operable to monitor the expired gases.

2. The oxygen mask of claim 1, wherein the oxygen inflow port is defined in a lateral portion of the mask body and generally adapted to direct oxygen flow toward the patient's nose and mouth.

3. The oxygen mask of claim 1, wherein the oxygen inflow port is laterally defined on the right side of the mask body and generally adapted to direct oxygen flow toward the patient's nose and mouth.

4. The oxygen mask of claim 1, wherein the oxygen inflow port is defined in a substantially central portion of the mask body proximately located near the patient's nose and mouth, and the L-shaped connector may swivel the oxygen conduit to the left or right of the patient.

5. The oxygen mask of claim 1, wherein the mask body further defines a nose portion and the oxygen inflow port is defined in the nose portion generally adapted to direct oxygen flow toward the patient's nose and mouth.

6. The oxygen mask of claim 1, further comprising:
a scope port defined in a substantially central portion of the mask body proximately located over the patient's mouth; and
a plug attached to the mask body with a strap adapted to substantially seal the scope port.

7. The oxygen mask of claim 1, further comprising a plurality of vent openings defined in the mask body.

8. The oxygen mask of claim 7, wherein the expired gas sampling tube has access to the compartment within the mask body via one of the plurality of vent openings.

9. The oxygen mask of claim 1, further comprising a nose clip adapted to better conform the mask to the patient's face.

10. A mask for delivering oxygen to a patient, comprising:
a mask body adapted to substantially cover at least the patient's nose and having a peripheral edge adapted to substantially seal against the patient's face and forming a compartment;
an oxygen inflow port defined in the mask body;
an oxygen conduit coupled to the oxygen inflow port and having access to the compartment in the mask body, the oxygen conduit adapted to conduct oxygen flow from an oxygen supply to the compartment in the mask body; and
an expired gas sampling tube coupled to the mask body and having access to the compartment in the mask body, the expired gas sampling tube having a perforated segment with a plurality of perforations and generally positioned proximate to the patient's nose and mouth, the expired gas sampling tube adapted to couple to an instrument operable to monitor the expired gases.

11. The oxygen mask of claim 10, wherein the oxygen inflow port is defined in a lateral portion of the mask body and generally adapted to direct oxygen flow toward the patient's nose and mouth.

12. The oxygen mask of claim 10, wherein the oxygen inflow port is laterally defined on the right side of the mask body and generally adapted to direct oxygen flow toward the patient's nose and mouth.

13. The oxygen mask of claim 10, wherein the oxygen inflow port is defined in a substantially central portion of the mask body proximately located near the patient's nose and mouth, and an L-shaped connector couples the oxygen conduit to the oxygen inflow port, the L-shaped connector may swivel the oxygen conduit to the left or right of the patient.

14. The oxygen mask of claim 10, wherein the mask body further defines a nose portion and the oxygen inflow port is defined in the mask body generally below and proximate the nostrils of the patient, the oxygen inflow port is adapted to direct oxygen flow toward the patient's nose and mouth, and an L-shaped connector couples the oxygen conduit to the oxygen inflow port.

15. The oxygen mask of claim 10, further comprising:
a scope port defined in a substantially central portion of the mask body proximately located over the patient's mouth; and
a plug adapted to substantially seal the scope port.

16. The oxygen mask of claim 10, further comprising a plurality of vent openings defined in the mask body.

17. The oxygen mask of claim 16, wherein the expired gas sampling tube has access to the compartment within the mask body via one of the plurality of vent openings.

18. The oxygen mask of claim 10, further comprising a nose clip adapted to better conform the mask to the patient's face.

19. The oxygen mask of claim 10, wherein the perforated segment of the expired gas sampling tube is attached to an interior surface of the mask body at at least one location.

20. An oxygen mask, comprising:
a mask body adapted to substantially cover a patient's nose and mouth and having a peripheral edge adapted to substantially seal against the patient's face and forming a compartment;
an oxygen inflow port defined in the mask body;
an oxygen conduit coupled to the mask body via the oxygen inflow port, the oxygen conduit adapted to conduct oxygen flow from an oxygen supply to the compartment in the mask body;
at least one vent opening defined laterally in the mask body; and
an expired gas sampling tube gaining access to the compartment in the mask body via the at least one lateral vent opening, the expired gas sampling tube having a perforated segment with a plurality of perforations and attached to an interior surface of the mask body proximate to the patient's nose and mouth, the expired gas sampling tube adapted to conduct expired gases from the patient to an instrument operable to make measurements of the expired gases.
